(19) United States
(12) Patent Application Publication
(10) Pub. No.: US 2019/0322632 A1
(43) Pub. Date: Oct. 24, 2019

(54) HRI ACTIVATORS USEFUL FOR THE TREATMENT OF CARDIOMETABOLIC DISEASES

(71) Applicant: UNIVERSITAT DE BARCELONA, Barcelona (ES)

(72) Inventors: Mohammad ZAREI, Barcelona (ES); Manuel VÁZQUEZ CARRERA, Barcelona (ES); Santiago VÁZQUEZ CRUZ, Cornellá de Llobregat (ES); Rosana LEIVA MARTÍNEZ, Barcelona (ES); Eugènia PUJOL BECH, Barcelona (ES)

(73) Assignee: Universitat de Barcelona, Barcelona (ES)

(21) Appl. No.: 16/317,485

(22) PCT Filed: Mar. 29, 2017
(86) PCT No.: PCT/EP2017/057398

§ 371 Date: Jan. 11, 2019

(30) Foreign Application Priority Data
Jul. 13, 2016 (EP) 16179237.9

(51) Int. Cl. C07D 285/14; C07C 381/00; A61P 1/16; A61P 3/08; C07D 271/12

(52) U.S. Cl. CPC C07D 285/14; C07C 381/00; C07D 271/12; A61P 3/08; A61P 1/16

(57) ABSTRACT

Compounds of formula (I) include: X is CH or N, preferably CH; n is 1-5, preferably 1-2; m is 0-5, preferably 1-2; and, when m is 2-5, two of the R² radicals taken together with two adjacent carbons of the benzene ring can form a 5- or 6-membered heterocyclic ring fused with the benzene ring. Compounds of formula (I) are heme-regulated inhibitor (HRI) activators and useful for prevention or treatment of cardiometabolic diseases such as metabolic syndrome, obesity, insulin resistance, type 2 diabetes mellitus, non-alcoholic fatty liver disease, steatosis, non-alcoholic steatohepatitis, hypertension, dyslipidemia, atherosclerosis, and heart disease.

### TECHNICAL FIELD

The present invention relates to the field of pharmaceutical chemistry, in particular to new compounds and their uses in the prevention or treatment of known diseases.

### BACKGROUND ART

Metabolic syndrome is a combination of conditions that occur together, increasing the risk in a person of cardiovascular diseases and diabetes. The conditions that may be encountered in metabolic syndrome are increased blood pressure, high blood sugar, excess body fat around the waist, a low HDL cholesterol level, and high triglyceride levels. You must have at least three metabolic risk factors to be diagnosed with metabolic syndrome. It is estimated that around 20-25% of the world's adult population have the metabolic syndrome. To date, although there are pharmaceutical medications for the treatment of diabetes, dyslipidaemia, obesity, and hypertension, no combined use appears to be satisfactory for the treatment of metabolic syndrome because of a poor efficacy, a limited tolerability, or both. In addition to the higher risk of adult population having the metabolic syndrome to have a cardiovascular disease such as a heart attack or a stroke, people with the mentioned combination of risk factors have a fivefold greater risk of developing type 2 diabetes. It is possible to prevent or delay metabolic syndrome, mainly having a healthy lifestyle. Nevertheless, successfully controlling metabolic syndrome requires long-term effort making some lifestyle changes and collaborating with health professionals. However, occasionally, the combination of a diet and of physic activity is insufficient to achieve the sought effect.

Available data from clinical, experimental and epidemiological studies indicate that non-alcoholic fatty liver disease (NAFLD) may be the hepatic manifestation of metabolic syndrome (cf. Marchesini G. et al., “Nonalcoholic fatty liver disease: a feature of the metabolic syndrome”, Diabetes, 2001, vol. 50, pp. 1844-1850). Studies over recent years have shown that NAFLD is associated with insulin resistance and diabetes, that NAFLD predicts the development of type 2 diabetes mellitus (T2DM) and vice versa, and that each condition serves as a progression factor for the other (cf. Musso G. et al., “Metaanalysis: natural history of non-alcoholic fatty liver disease (NAFLD) and diagnostic accuracy of non-invasive tests for liver disease severity”, Ann. Med., 2011, vol. 43, pp. 617-649). On the other hand, NAFLD incorporates a spectrum of pathology from simple steatosis to non-alcoholic steatohepatitis (NASH), to fibrosis and cirrhosis (cf. Musso G. et. al., ibid.). Significant steatosis is defined as fat (triglyceride) accumulation in more than 5% of hepatocytes. Insulin resistance is a key pathogenic factor in both NAFLD and metabolic syndrome.

It is known that HRI activators induce eIF2α phosphorylation, reducing the abundance of eIF2.GTP.tRNAᵢ^{Met} ternary complex, and thus inhibiting cancer cell proliferation (cf. Denoyelle S. et al., “In vitro inhibition of translation initiation by N,N′-diarylureas potential anti-cancer agents”, Bioorganic & Medicinal Chemistry Letters, 2012, vol. 22, pp. 402-409). Particularly, some N,N′-diarylureas have been identified as agents that activate HRI and, as such, these agents inhibit the proliferation of certain cancer cells, thus being potential anti-cancer agents (cf. Chen T. et al., “Chemical genetics identify eIF2α kinase heme-regulated inhibitor as an anticancer target”, Nature Chemical Biology, 2011, vol. 7, pp. 610-616, and Supporting Information).

Currently, there is no unique long-term effective pharmaceutical treatment for the metabolic syndrome. Thus, there is still a need for developing compounds showing improved activity in the treatment of conditions related to metabolic syndrome and its associated disorders, such as obesity and T2DM, as well as other cardiometabolic diseases related with fat accumulation such as NAFLD and NASH.

### SUMMARY OF INVENTION

An aspect of the present invention is the provision of compounds of formula (I)

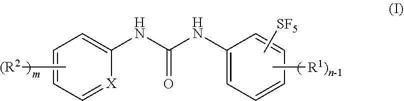

and pharmaceutically acceptable salts and solvates thereof, wherein: X is CH or N;

R¹ is a radical independently selected from the group consisting of H, SF₅, halogen, CF₃, NO₂, CN, and OCF₃; n being an integer from 1 to 5; halogen being F, Cl, Br or I;

R² is a radical independently selected from the group consisting of SF₅, halogen, CF₃, NO₂, CN, OCF₃, hydroxyl, (C₁-C₄)alkyl, (C₁-C₄)alkoxy, di-(C₁-C₄)alkylaminoethoxy, 2-(piperidin-1-yl)ethoxy, 2-(pyrrolidin-1-yl)ethoxy, 2-(azepan-1-yl)ethoxy, 2-morpholinoethoxy, and 2-(piperazin-1-yl)ethoxy; m being an integer from 0 to 5;

alternatively, when X is CH and m is an integer from 2 to 5, two R² radicals taken together with two adjacent carbons of the benzene ring to which they are joined form a 5- or 6-membered heterocyclic ring having from 1 to 3 heteroatoms independently selected from the group consisting of O, N, and S, the heterocyclic ring being fused with the benzene ring, and the benzene ring being optionally substituted with one or more radicals independently selected from the group consisting of SF₅, halogen, CF₃, NO₂, CN, and OCF₃;

provided that the compound of formula (I) does not have any of the following formulas:

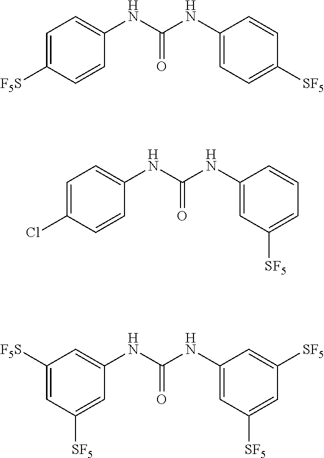

The three N,N′-diarylureas disclaimed from formula (I) are chemically disclosed: the first one as a synthetic intermediate (c.f. Karagiannidis L. E. et al., “Highly effective yet simple transmembrane anion transporter based upon ortho-phenylenediamine bis-ureas”, Chem. Commun., 2014. vol. 50, pp. 12050-12053, Electronic Supplementary Information), and the other two as having activity in algal control on soil and in water, bacterial control, expulsion of intestinal worms, inhibition of chlorophyll formation in plants and selective weed control (cf. U.S. Pat. No. 3,073,861).

In a particular embodiment compounds of formula (I) have X=CH. In another particular embodiment, in compounds of formula (I) n is 1 or 2, and m is 1 or 2. Particular embodiments are those in which in formula (I) an SF₅ radical is attached to the 3 position of the phenyl ring that has the R¹ radicals; also those where halogen is F or Cl.

In a particular embodiment of compounds (I), m is an integer from 2 to 5, and two R² radicals taken together with two adjacent carbons of the benzene ring to which they are joined form a 5- or 6-membered heterocyclic ring having from 1 to 3 heteroatoms independently selected from the group consisting of O, N, and S, the heterocyclic ring being fused with the benzene ring, and the benzene ring being optionally substituted with one or more groups independently selected from the group consisting of SF₅, halogen, CF₃, NO₂, CN, and OCF₃. More particular embodiments are those wherein the two R² radicals are forming a heterocyclic ring which has one of the following formulas:

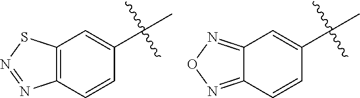 [SMILES: *c1ccc2nnsc2c1 ; *c1ccc2nonc2c1]

Even more particular, are those embodiments where compound (I) is selected from the group consisting of the following compounds, whose preparation is disclosed in the accompanying examples: 1-(benzo[d][1,2,3]thiadiazol-6-yl)-3-(4-(pentafluoro-λ⁶-sulfanyl)phenyl)urea (compound I-25); 1-(benzo[d][1,2,3]thiadiazol-6-yl)-3-(3-(pentafluoro-λ⁶-sulfanyl)phenyl)urea (compound I-26); 1-(benzo[d][1,2,3]thiadiazol-6-yl)-3-(2-chloro-5-(pentafluoro-λ⁶-sulfanyl)phenyl)urea (compound I-27); 1-(benzo[d][1,2,3]thiadiazol-6-yl)-3-(2-chloro-3-(pentafluoro-λ⁶-sulfanyl)phenyl)urea (compound I-28); and 1-(benzo[d][1,2,3]thiadiazol-6-yl)-3-(4-chloro-3-(pentafluoro-λ⁶-sulfanyl)phenyl)urea (compound I-29).

In another particular embodiment compounds (I) are those where R² is a radical independently selected from the group consisting of SF₅, halogen, CF₃, NO₂, CN, OCF₃, hydroxyl, (C₁-C₄)alkyl, (C₁-C₄)alkoxy, di-(C₁-C₄)alkylaminoethoxy, 2-(piperidin-1-yl)ethoxy, 2-(pyrrolidin-1-yl)ethoxy, 2-(azepan-1-yl)ethoxy, 2-morpholinoethoxy, and 2-(piperazin-1-yl)ethoxy. More particular are those embodiments where compound (I) is selected from the group consisting of the following compounds, whose preparation is disclosed in the accompanying examples: 1,3-bis(2-chloro-5-(pentafluoro-λ⁶-sulfanyl)phenyl)urea (compound I-32); 1-(4-chloro-3-(trifluoromethyl)phenyl)-3-(2-chloro-5-(pentafluoro-λ⁶-sulfanyl)phenyl)urea (compound I-33); 1,3-bis(3-(pentafluoro-λ⁶-sulfanyl)phenyl)urea (compound I-36); 1-(3-(pentafluoro-λ⁶-sulfanyl)phenyl)-3-(4-(pentafluoro-λ⁶-sulfanyl)phenyl)urea (compound I-37); and 1,3-bis(4-chloro-3-(pentafluoro-λ⁶-sulfanyl)phenyl)urea (compound I-38).

Inventors have found that the compounds of the present invention are HRI activators, and therefore that they are useful as active pharmaceutical ingredients, specifically in the prevention or treatment of those metabolic diseases that may be called ‘cardiometabolic diseases’, such as metabolic syndrome, obesity, insulin resistance, type 2 diabetes mellitus, non-alcoholic fatty liver disease, steatosis, non-alcoholic steatohepatitis, hypertension, dyslipidemia, atherosclerosis, and heart disease, particularly the metabolic syndrome. Thus, another aspect of the present invention relates the compounds of the present invention for use in the prevention or therapy of those diseases. This aspect may also be expressed as a method of prevention or treatment of a human or animal patient suffering from some of those diseases, comprising the administration of a therapeutically effective amount of a compound of the present invention. It can also be expressed as the use of a compound for the preparation of a medicine for the prevention or treatment of the those diseases.

Another aspect of the invention relates to pharmaceutical compositions comprising a therapeutically effective amount of a compound of the present invention, together with appropriate amounts of pharmaceutically acceptable excipients or carriers. These compositions can be used in the prevention or treatment of the above-mentioned cardiometabolic diseases.

Compounds of formula (I) can be obtained by reacting an isocyanate of formula (III) with an amine derivative of formula (II), wherein radicals have the above-defined values. Optionally, the reaction is carried out in the presence of a base.

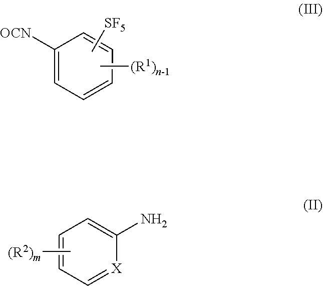

In a preferred embodiment the compound of formula (II) is first deprotonated with a suitable base, preferably n-butyllithium, in an anhydrous solvent as tetrahydrofuran, preferably at low temperature, and then the compound of formula (III) is added. Alternatively, the reaction is carried out in the absence of base, preferably at room temperature. In turn, the isocyanate of formula (III) is commercially available or can be obtained by the reaction of a suitable amine of formula (IV) with triphosgene in the presence of a base such a triethylamine, in an organic solvent such as toluene.

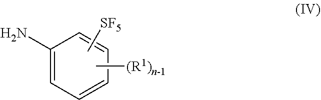

Scheme 1 and Scheme 2 illustrate general preparation processes of the compounds of the invention. The commercial availability of aniline and isocyanate derivatives to carry out the preparation processes are illustrated in Table 1 and Table 2, respectively.

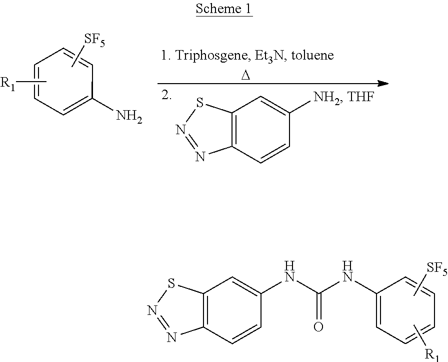

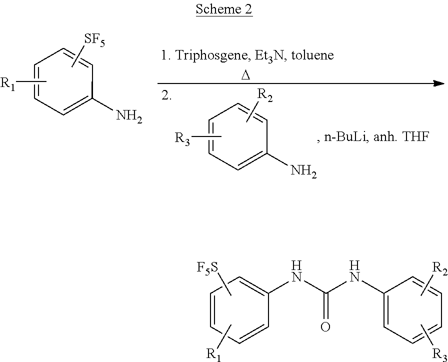

TABLE 1

| Aniline derivative (number) | Source |
|---|---|
| 4-(pentafluoro-λ⁶-sulfanyl)aniline (1) | commercially available |
| 3-(pentafluoro-λ⁶-sulfanyl)aniline (2) | commercially available |
| 2-chloro-5-(pentafluoro-λ⁶- | chlorination of aniline (2) |
| sulfanyl)aniline (3) | cf. U.S. Pat. No. 7,932,416, |
| | Example 3 |
| 2-chloro-3-(pentafluoro-λ⁶- | chlorination of aniline (2) |
| sulfanyl)aniline (4) | cf. U.S. Pat. No. 7,932,416, |
| | Example 3 |
| 4-chloro-3-(pentafluoro-λ⁶- | chlorination of aniline (2). |
| sulfanyl)aniline (5) | cf. U.S. Pat. No. 7,932,416, |
| | Example 3 |
| benzo[d][1,2,3]thiadiazol-6-amine (6) | cf. Step 3 of the Supporting |
| | Information of Chen T. et. al., |
| | ibid. |
| 4-chloro-3-(trifluoromethyl)aniline (7) | commercially available |

TABLE 2

| Isocyanate derivative | Source |
|---|---|
| 4-(pentafluoro-λ⁶-sulfanyl)phenyl | from aniline (1) |
| isocyanate | cf. U.S. Pat. No. 8,937,088, |
| | Example 1 |
| 3-(pentafluoro-λ⁶-sulfanyl)phenyl | from aniline (2) |
| isocyanate | cf. U.S. Pat. No. 8,937,088, |
| | Example 1 |
| 2-chloro-5-(pentafluoro-λ⁶-sulfanyl)phenyl | from aniline (3) |
| isocyanate | cf. U.S. Pat. No. 8,937,088, |
| | Example 1 |
| 2-chloro-3-(pentafluoro-λ⁶-sulfanyl)phenyl | from aniline (4) |
| isocyanate | cf. U.S. Pat. No. 8,937,088, |
| | Example 1 |
| 4-chloro-3-(pentafluoro-λ⁶-sulfanyl)phenyl | from aniline (6) |
| isocyanate | cf. U.S. Pat. No. 8,937,088, |
| | Example 1 |

The preparation of pharmaceutically acceptable salts of compounds of formula (I) can be carried out by methods known in the art. For instance, they can be prepared from the parent compound, which contains a basic or acidic moiety, by conventional chemical methods. Generally, such salts are, for example, prepared by reacting the free acid or base forms of these compounds with a stoichiometric amount of the appropriate pharmaceutically acceptable base or acid, respectively, in water or in an organic solvent, or in a mixture of them. The compounds of formula (I) and their salts may differ in some physical properties but they are equivalent for the purposes of the present invention.

The compounds of the invention may be in crystalline form, either as free solvation compounds or as solvates (e.g. hydrates), and it is intended that all these forms are within the scope of the present invention. Methods of solvation are generally known within the art. In general, the solvated forms with pharmaceutically acceptable solvents such as water, or ethanol, are equivalent to the unsolvated form for the purposes of the invention.

As demonstrated in the examples, HRI activators are useful to ameliorate several metabolic parameters such as plasma glucose and triglyceride levels and hepatic steatosis. Thus, they may be used in the therapeutic or prophylactic treatment of cardiometabolic diseases such as metabolic syndrome, obesity, insulin resistance, type 2 diabetes mellitus, non-alcoholic fatty liver disease, steatosis, non-alcoholic steatohepatitis, hypertension, dyslipidemia, atherosclerosis, and heart disease.

Throughout the description and claims, the word “comprise” and variations of the word are not intended to exclude other technical features, additives, components, or steps. Furthermore, the word “comprise” encompasses the case of “consisting of”. The following examples are illustrative and not intended to be limiting of the present invention.

### BRIEF DESCRIPTION OF DRAWINGS

FIG. 1 shows immunoblot analyses and the quantification of total and phospho-HRI in human Huh-7 hepatic cells incubated for 24 h with vehicle (DMSO, CT cells) or 10 μM of each of compounds I-25, I-26, I-27, I-28, and I-29. Data are presented as the mean±SD (n=4). *p<0.05 vs. control (CT) cells. ^{#}p<0.05 vs. BTdCPU-treated cells. BTdCPU=1-(benzo[d][1,2,3]thiadiazol-6-yl)-3-(3,4-dichlorophenyl)urea (compound (2) in Chen T. et al., ibid.).

FIG. 2 shows immunoblot analyses and the quantification of total and phospho-HRI in human Huh-7 hepatic cells incubated for 24 h with vehicle (DMSO, CT cells) or 10 μM of each of compounds I-30, I-2, and I-33. Data are presented as the mean±SD (n=4). ***p<0.001 and *p<0.05 vs. control (CT) cells. ^{#}p<0.05 vs. BTdCPU-treated cells. Compound I-2 (1-(4-chloro-3-(trifluoromethyl)phenyl)-3-(4-chloro-3-(trifluoromethyl)phenyl)urea is not part of the present invention, but it has been introduced in some figures for comparative purposes.

FIG. 3 shows immunoblot analyses and the quantification of total and phospho-eIF2α in human Huh-7 hepatic cells incubated for 24 h with vehicle (DMSO, CT cells) or 10 μM of each of compounds I-25, I-26, I-27, I-28 and I-29. Data are presented as the mean±SD (n=4). **p<0.01 and *p<0.05 vs. control (CT) cells. ^{##}p<0.01 and ^{#}p<0.05 vs. BTdCPU-treated cells.

FIG. 4 shows immunoblot analyses and the quantification of total and phospho-eIF2α in human Huh-7 hepatic cells incubated for 24 h with vehicle (DMSO, CT cells) or 10 μM of each of compounds I-30 and I-33. Data are presented as the mean±SD (n=4). *p<0.05 vs. control (CT) cells.

FIG. 5 shows the macroscopic (upper image) and microscopic images of Huh-7 cells stained with Oil Red O. Huh-7 cells were previously incubated for 24 h with BSA (Control, CT), 0.75 mmol/L palmitate (Pal) conjugated with BSA, or 0.75 mmol/L BSA-palmitate plus 10 μmol/L BTdCPU (Pal+BTdCPU).

FIG. 6 shows the immunoblot analyses of total and phosphorylated Akt (A), and total Akt. (B). When indicated (+), cells were incubated with 100 nmol/L insulin (I) for the last 10 min. Data are presented as the mean±SD (n=4 per group). ***p<0.001 and *p<0.05 vs. control cells not exposed to insulin. ^{###}p<0.001 and ^{##}p<0.01 vs. insulin-stimulated control cells. ^{554 \\}p<0.001 vs. insulin-stimulated cells incubated with palmitate. Ins: insulin.

FIG. 7 shows the results of the glucose tolerance test and area under the curve (AUC) of mice fed a standard chow (CT), a HFD for three weeks (HFD), or a HFD for three weeks plus BTdCPU during the last week (HFD+BTdCPU). Data are presented as the mean±SD (n=6 per group). *p<0.05 vs. mice fed a standard diet (CT). #p<0.05 vs. mice fed a HFD. G: glucose.

FIG. 8. (A) Oil Red O and eosin-hematoxylin (H&E) staining of livers of mice fed a standard chow (CT), a HFD for three weeks (HFD) or a HFD for three weeks plus BTdCPU during the last week (HFD+BTdCPU). (B) Liver triglyceride levels. Data are presented as the mean±SD (n=6 per group). *p<0.05 vs. mice fed a standard diet (CT). ^{#}p<0.05 vs. mice fed a HFD. G: glucose. TG: triglyceride.

FIG. 9 shows the effects of different HRI activators on FGF21 expression in hepatocytes. Human Huh-7 hepatocytes were incubated for 24 h in the absence (Control, CT) or in the presence of compounds (10 μM). Assessment by quantitative real-time RT-PCR of FGF21. Data are presented as the mean±SD (n=4 per group). ***p<0.001 and **p<0.01 vs CT. ^{###}p<0.001, ^{##}p<0.01 and ^{#}p<0.05 vs BTCtFPU. ^{††}p<0.01 and ^{†}p<0.05 vs BTdCPU. BTCtFPU=1-(benzo[d][1,2,3]thiadiazol-6-yl)-3-(4-chloro-3-(trifluoromethyl)phenyl)urea (compound (3) in Chen T. et al., ibid.).

### DESCRIPTION OF EMBODIMENTS

### Preparative Example 1. 1-(Benzo[d][1,2,3]thiadiazol-6-yl)-3-(4-(pentafluoro-λ⁶-sulfanyl)phenyl)urea (compound I-25). Step 1

A solution of 4-(pentafluoro-λ⁶-sulfanyl)aniline (259 mg, 1.18 mmol) in toluene (5 mL) was treated with triphosgene (175 mg, 0.59 mmol). Immediately, triethylamine (0.16 mL, 1.18 mmol) was added and the reaction mixture was stirred at 70° C. for 2 h. Afterwards, pentane (1 mL) was added and a white precipitate was formed. The mixture was filtered and pentane was evaporated in vacuo at room temperature to give 4-(pentafluoro-λ⁶-sulfanyl)phenyl isocyanate in toluene solution that was used in the next step without further purification. Step 2. To a solution of the isocyanate from the previous step a solution of benzo[d][1,2,3]thiadiazol-6-amine (178 mg, 1.18 mmol) in THF (6 mL) was added. The suspension was stirred at room temperature overnight. Evaporation in vacuo of the organics gave an orange solid. Column chromatography (hexane/ethyl acetate mixtures) gave compound I-25 as a white solid (203 mg, 43% overall yield). The analytical sample was obtained by washing with cooled dichloromethane (126 mg). m.p.: 274-275° C. IR (ATR) v: 612, 641, 659, 716, 801, 832, 1059, 1101, 1134, 1191, 1245, 1271, 1297, 1325, 1351, 1413, 1467, 1510, 1528, 1595, 1721, 3100, 3136, 3297, 3338 cm⁻¹. Accurate mass: Calculated for [C₁₃H₉F₅N₄OS₂—H]⁻: 395.0065; Found: 395.0064.

### Preparative Example 2. 1-(Benzo[d][1,2,3]thiadiazol-6-yl)-3-(3-(pentafluoro-λ⁶-sulfanyl)phenyl)urea (compound I-26). Step 1

A solution of 3-(pentafluoro-λ⁶-sulfanyl)aniline (259 mg, 1.18 mmol) in toluene (5.8 mL) was treated with triphosgene (175 mg, 0.59 mmol). Immediately, triethylamine (0.16 mL, 1.18 mmol) was added and the reaction mixture was stirred at 70° C. for 2 h. Afterwards, pentane (1 mL) was added and a white precipitate was formed. The mixture was filtered and pentane was evaporated in vacuo at room temperature to give 3-(pentafluoro-λ⁶-sulfanyl)phenyl isocyanate in toluene solution that was used in the next step without further purification. Step 2. To a solution of the isocyanate from the previous step a solution of benzo[d][1,2,3]thiadiazol-6-amine (178 mg, 1.18 mmol) in THF (6 mL) was added. The suspension was stirred at room temperature overnight. Evaporation in vacuo of the organics gave an orange solid (440 mg). Column chromatography (hexane/ethyl acetate mixtures) gave compound I-26 as a pale white solid (243 mg, 52% overall yield). The analytical sample was obtained by washing with pentane (199 mg). m.p.: 229-230 OC. IR (ATR) v: 614, 680, 718, 780, 801, 819, 830, 878, 912, 922, 1057, 1009, 1113, 1134, 1196, 1219, 1294, 1312, 1349, 1412, 1434, 1466, 1529, 1540, 1568, 1602, 1718, 2852, 2919, 3090, 3126, 3271, 3302, 3338 cm⁻¹. Elemental analysis: Calculated for C₁₃H₉F₅N₄OS₂. 0.05C₄H₈O₂.0.5C₅H₁₂: C, 43.17%, H, 3.55%, N, 12.83%, S, 14.68%; Found: C, 43.51%, H, 3.17%, N, 12.96%, S, 14.61%.

### Preparative Example 3. 1-(Benzo[d][1,2,3]thiadiazol-6-yl)-3-(2-chloro-5-(pentafluoro-λ⁶-sulfanyl)phenyl)urea (compound I-27). Step 1

A solution of 2-chloro-5-(pentafluoro-λ⁶-sulfanyl)aniline (300 mg, 1.18 mmol) in toluene (5 mL) was treated with triphosgene (175 mg, 0.59 mmol). Immediately, triethylamine (0.16 mL, 1.18 mmol) was added and the reaction mixture was stirred at 70° C. for 2 h. Afterwards, pentane (1 mL) was added and a white precipitate was formed. The mixture was filtered and pentane was in vacuo at room temperature to give 2-chloro-5-(pentafluoro-λ⁶-sulfanyl)phenyl isocyanate in toluene solution that was used in the next step without further purification. Step 2. To a solution of the isocyanate from the previous step a solution of benzo[d][1,2,3]thiadiazol-6-amine (178 mg, 1.18 mmol) in THF (6 mL) was added. The suspension was stirred at room temperature overnight. Evaporation in vacuo of the organics gave an orange solid (378 mg). Column chromatography (hexane/ethyl acetate mixtures) gave compound I-27 as a beige solid (106 mg, 21% overall yield). The analytical sample was obtained by washing with cooled dichloromethane (80 mg). m.p.: 227° C. IR (ATR) v: 615, 664, 729, 805, 816, 837, 884, 922, 964, 1033, 1052, 1134, 1222, 1287, 1349, 1413, 1467, 1536, 1563, 1666, 1721, 3333 cm⁻¹. Accurate mass: Calculated for [C₁₃H₈ClF₅N₄OS₂—H]⁻: 428.9675; Found: 428.9676.

### Preparative Example 4. 1-(Benzo[d][1,2,3]thiadiazol-6-yl)-3-(2-chloro-3-(pentafluoro-λ⁶-sulfanyl)phenyl)urea (compound I-28)

Step 1. A solution of 2-chloro-3-(pentafluoro-λ⁶-sulfanyl)aniline (300 mg, 1.18 mmol) in toluene (5 mL) was treated with triphosgene (175 mg, 0.59 mmol). Immediately, triethylamine (0.16 mL, 1.18 mmol) was added and the reaction mixture was stirred at 70° C. for 2 h. Afterwards, pentane (1 mL) was added and a white precipitate was formed. The mixture was filtered and pentane was evaporated in vacuo at room temperature to give 2-chloro-3-(pentafluoro-λ⁶-sulfanyl)phenyl isocyanate in toluene solution that was used in the next step without further purification. Step 2. To a solution of the isocyanate from the previous step a solution of benzo[d][1,2,3]thiadiazol-6-amine (178 mg, 1.18 mmol) in THF (6 mL) was added. The suspension was stirred at room temperature overnight. Evaporation in vacuo of the organics gave an orange solid (478 mg). Column chromatography (hexane/ethyl acetate mixtures) gave compound I-28 as a pale white solid (149 mg, 30% overall yield). m.p.: 225° C. IR (ATR) v: 628, 649, 673, 705, 729, 760, 783, 814, 847, 915, 1054, 1126, 1155, 1217, 1245, 1279, 1318, 1346, 1411, 1462, 1527, 1571, 1664, 1718, 2852, 2919, 2956, 3317 cm⁻¹. Elemental analysis: Calculated for C₁₃H₈ClF₅N₄OS₂.0.05C₄H₈O₂.0.5C₆H₁₄: C, 40.68%, H, 3.25%, N, 11.71%, S, 13.41%; Found: C, 40.88%, H, 3.00%, N, 11.51%, S, 13.17%.

### Preparative Example 5. 1-(Benzo[d][1,2,3]thiadiazol-6-yl)-3-(4-chloro-3-(pentafluoro-λ⁶-sulfanyl)phenyl)urea (compound I-29). Step 1

A solution of 4-chloro-3-(pentafluoro-λ⁶-sulfanyl)aniline (300 mg, 1.18 mmol) in toluene (5 mL) was treated with triphosgene (175 mg, 0.59 mmol). Immediately, triethylamine (0.16 mL, 1.18 mmol) was added and the reaction mixture was stirred at 70° C. for 2 h. Afterwards, pentane (1 mL) was added and a white precipitate was formed. The mixture was filtered and pentane was evaporated in vacuo at room temperature to give 4-chloro-3-(pentafluoro-λ⁶-sulfanyl)phenyl isocyanate in toluene solution that was used in the next step without further purification. Step 2. To a solution of the isocyanate from the previous step a solution of benzo[d][1,2,3]thiadiazol-6-amine (178 mg, 1.18 mmol) in THF (6 mL) was added. The suspension was stirred at room temperature overnight. Evaporation in vacuo of the organics gave an orange solid (325 mg). Column chromatography (hexane/ethyl acetate mixtures) gave compound I-29 as a pale brown solid (237 mg, 47% overall yield). The analytical sample was obtained by washing with pentane (180 mg). m.p.: 213-214° C. IR (ATR) v: 647, 669, 726, 801, 819, 845, 894, 923, 960, 1033, 1126, 1152, 1207, 1245, 1276, 1315, 1349, 1387, 1413, 1465, 1527, 1571, 1594, 1723, 2919, 3091, 3126, 3178, 3271, 3297, 3338 cm⁻¹. Elemental analysis: Calculated for C₁₃H₈ClF₅N₄OS₂. 0.5C₆H₁₄.0.25C₄H₈O₂: C, 41.17%, H, 3.46%, N, 11.30%, S, 12.93%; Found: C, 41.30%, H, 3.22%, N, 11.43%, S, 12.72%.

### Preparative Example 6. 1,3-Bis(2-chloro-5-(pentafluoro-λ⁶-sulfanyl)phenyl)urea, (Compound I-32). Step 1

A solution of 2-chloro-5-(pentafluoro-λ⁶-sulfanyl)aniline (350 mg, 1.38 mmol) in toluene (4 mL) was treated with triphosgene (204 mg, 0.69 mmol). Immediately, triethylamine (0.19 mL, 1.38 mmol) was added and the reaction mixture was stirred at 70° C. for 2 h. Afterwards, pentane (1 mL) was added and a white precipitate was formed. The mixture was filtered and pentane was evaporated in vacuo at room temperature to give 2-chloro-5-(pentafluoro-λ⁶-sulfanyl)phenyl isocyanate in toluene solution that was used in the next step without further purification. Step 2. 2-chloro-5-(pentafluorosulfanyl)aniline (384 mg, 1.51 mmol) was dissolved in anh. THF (12 mL) under argon and cooled to −78° C. on a dry ice in acetone bath. Then, 2.5 M n-butyllithium in hexanes (0.73 mL, 1.78 mmol) was added dropwise during 20 min. Afterwards, the reaction mixture was removed from the dry ice in acetone bath and tempered to 0° C. with an ice bath. Meanwhile, the isocyanate (387 mg, 1.38 mmol) in toluene solution from the previous step was stirred under argon and was continuously added to the reaction mixture. The mixture was stirred at room temperature overnight. Methanol (4.5 mL) was added to quench any unreacted n-butyllithium and evaporation of the solvents provided a pale brown solid (670 mg). Column chromatography (hexane/ethyl acetate mixtures) gave compound I-32 as a beige solid (283 mg, 39% overall yield). m.p.: 227-228° C. IR (ATR) v: 663, 703, 736, 752, 798, 805, 820, 887, 920, 961, 1037, 1075, 1108, 1157, 1233, 1256, 1281, 1294, 1414, 1460, 1533, 1587, 1661, 1696, 1717, 1890, 1908, 2843, 2920, 2956, 3294, 3304, 3329 cm⁻¹. Accurate mass: Calculated for [C₁₃H₈Cl₂F₁₀N₂OS₂—H]⁻: 530.9223; Found: 530.9222.

### Preparative Example 7. 1-(4-Chloro-3-(trifluoromethyl)phenyl)-3-(2-chloro-5-(pentafluoro-λ⁶-sulfanyl)phenyl)urea (compound I-33). Step 1

A solution of 2-chloro-5-(pentafluoro-λ⁶-sulfanyl)aniline (350 mg, 1.38 mmol) in toluene (4 mL) was treated with triphosgene (204 mg, 0.69 mmol) under argon. Immediately, triethylamine (0.19 mL, 1.38 mmol) was added and the reaction mixture was stirred at 70° C. for 2 h. Afterwards, pentane (1 mL) was added and a white precipitate was formed. The mixture was filtered and pentane was evaporated in vacuo at room temperature to give 2-chloro-5-(pentafluoro-λ⁶-sulfanyl)phenyl isocyanate in toluene solution that was used in the next step without further purification. Step 2. 4-chloro-3-(trifluoromethyl)aniline (296 mg, 1.51 mmol) was dissolved in anh. THF (12 mL) under argon and cooled to −78° C. on a dry ice in acetone bath. Then, 2.5 M n-butyllithium in hexanes (0.73 mL, 1.78 mmol) was added dropwise during 20 min. Afterwards, the reaction mixture was removed from the dry ice in acetone bath and tempered to 0° C. with an ice bath. Meanwhile, the isocyanate (387 mg, 1.38 mmol) in toluene solution from the previous step was stirred under argon and was continuously added to the reaction mixture. The mixture was stirred at room temperature overnight. Methanol (5 mL) was added to quench any unreacted n-butyllithium and evaporation of the solvents provided a brown oil (767 mg). Column chromatography (hexane/ethyl acetate mixtures) gave compound I-33 as a pale brown solid (138 mg, 22% overall yield). m.p.: 156-157° C. IR (ATR) v: 632, 666, 684, 701, 727, 742, 760, 801, 812, 840, 855, 863, 906, 950, 963, 1034, 1065, 1111, 1126, 1175, 1216, 1229, 1260, 1283, 1301, 1329, 1372, 1408, 1459, 1485, 1513, 1546, 1582, 1592, 1608, 1654, 1695, 1715, 1769, 1905, 1925, 2025, 2179, 2323, 2369, 2851, 2917, 2953, 3276, 3328, 3671, 3733, 3795, 3815 cm⁻¹. Accurate mass: Calculated for [C₁₄H₈Cl₂F₈N₂OS—H]⁻: 475.9534; Found: 475.9538.

### Preparative Example 8. 1,3-Bis(3-(pentafluoro-λ⁶-sulfanyl)phenyl)urea (compound I-36). Step 1

A solution of 3-(pentafluoro-λ⁶-sulfanyl)aniline (350 mg, 1.60 mmol) in toluene (6.8 mL) was treated with triphosgene (237 mg, 0.80 mmol). Immediately, triethylamine (0.22 mL, 1.60 mmol) was added and the reaction mixture was stirred at 70° C. for 2 h. After, pentane (1 mL) was added and a white precipitate was formed. The mixture was filtered and pentane was evaporated in vacuo at room temperature to give 3-(pentafluoro-λ⁶-sulfanyl)phenyl isocyanate in toluene solution that was used in the next step without further purification. Step 2. 3-(Pentafluoro-λ⁶-sulfanyl)aniline (351 mg, 1.60 mmol) was dissolved in anh. THF (3 mL) under argon and cooled to −78° C. on a dry ice in acetone bath. Then, 2.5 M n-butyllithium in hexanes (0.86 mL, 2.08 mmol) was added dropwise during 20 min. Afterwards, the reaction mixture was removed from the dry ice in acetone bath and tempered to 0° C. with an ice bath. Meanwhile, the isocyanate (392 mg, 1.60 mmol) from previous step was stirred under argon and was continuously added to the reaction mixture. The mixture was stirred at room temperature overnight. Methanol (4.5 mL) was added to quench any unreacted n-butyllithium, and evaporation of solvents provided a beige solid (710 mg). Column chromatography (hexane/ethyl acetate mixtures) gave I-36 as a pale white solid (362 mg, 49% overall yield). The analytical sample was obtained as a white solid (183 mg) by crystallization from hot ethyl acetate. m.p.: 267-268° C. IR (ATR) v: 1117, 1242, 1314, 1418, 1485, 1599, 1663, 3102, 3202, 3310 cm⁻¹. Accurate mass: Calculated for [C₁₃H₁₀F₁₀N₂OS₂—H]⁻: 463.0002; Found: 463.0022.

### Preparative Example 9. 1-(3-(Pentafluoro-λ⁶-sulfanyl)phenyl)-3-(4-(pentafluoro-λ⁶-sulfanyl)phenyl)urea (compound I-37). Step 1

A solution of 3-(pentafluoro-λ⁶-sulfanyl)aniline (350 mg, 1.60 mmol) in toluene (5 mL) was treated with triphosgene (237 mg, 0.80 mmol). Immediately, triethylamine (0.22 mL, 1.60 mmol) was added and the reaction mixture was stirred at 70° C. for 2 h. Afterwards, pentane (1 mL) was added and a white precipitate was formed. The mixture was filtered and pentane was evaporated in vacuo at room temperature to give 3-(pentafluoro-λ⁶-sulfanyl)phenyl isocyanate in toluene solution that was used in the next step without further purification. Step 2. 4-(Pentafluoro-λ⁶-sulfanyl)aniline (246 mg, 1.12 mmol) was dissolved in anh. THF (5 mL) under argon and cooled to −78° C. on a dry ice in acetone bath. Then, 2.5 M n-butyllithium in hexanes (0.6 mL, 1.46 mmol) was added dropwise during 20 min. Afterwards, the reaction mixture was removed from the dry ice in acetone bath and tempered to 0° C. with an ice bath. Meanwhile, the isocyanate (275 mg, 1.12 mmol) from the previous step was stirred under argon and was continuously added to the reaction mixture. The mixture was stirred at room temperature overnight. Methanol (4.5 mL) was added to quench any unreacted n-butyllithium, and evaporation of the solvents provided a brown gum (742 mg). Column chromatography (hexane/ethyl acetate mixtures) gave compound I-37 as a pale white solid (102 mg, 20% overall yield). m.p.: 216-217° C. IR (ATR) v: 1103, 1196, 1229, 1304, 1410, 1487, 1549, 1597, 1665, 3088, 3134, 3204, 3321 cm⁻¹. Accurate mass: Calculated for [C₁₃H₁₀F₁₀N₂OS₂—H]⁻: 463.0002. Found: 463.0017.

### Preparative Example 10. 1,3-Bis(4-chloro-3-(pentafluoro-λ⁶-sulfanyl)phenyl)urea (Compound I-38). Step 1

A solution of 4-chloro-3-(pentafluoro-λ⁶-sulfanyl)aniline (350 mg, 1.37 mmol) in toluene (5 mL) was treated with triphosgene (204 mg, 0.69 mmol). Immediately, triethylamine (0.20 mL, 1.37 mmol) was added and the reaction mixture was stirred at 70° C. for 2 h. Afterwards, pentane (1 mL) was added and a white precipitate was formed. The mixture was filtered and pentane was evaporated in vacuo at room temperature to give 4-chloro-3-(pentafluoro-λ⁶-sulfanyl)phenyl isocyanate in toluene solution that was used in the next step without further purification. Step 2. 4-chloro-3-(pentafluoro-λ⁶-sulfanyl)aniline (278 mg, 1.09 mmol) was dissolved in anh. THF (6 mL) under argon and cooled to −78° C. on a dry ice in acetone bath. Then, 2.5 M n-butyllithium in hexanes (0.60 mL, 1.42 mmol) was added dropwise during 20 min. Afterwards, the reaction mixture was removed from the dry ice in acetone bath and tempered to 0° C. with an ice bath. Meanwhile, the isocyanate (307 mg, 1.09 mmol) from the previous step was stirred under argon and was continuously added to the reaction mixture. The mixture was stirred at room temperature overnight. Methanol (4.5 mL) was added to quench any unreacted n-butyllithium, and evaporation of the solvents provided an orange gum (675 mg). Column chromatography (hexane/ethyl acetate mixtures) gave compound I-38 as a white solid (136 mg, 23% overall yield). m.p.: 237-238° C. IR (ATR) v: 1042, 1130, 1227, 1290, 1396, 1477, 1545, 1587, 1645, 1699, 3030, 3138, 3306 cm⁻¹. Accurate mass: Calculated for [C₁₃H₈Cl₂F₁₀N₂OS₂—H]⁻: 530.9223. Found: 530.9236.

Identification and Activity Examples of HRI Activators

The following abbreviations are used: Akt: Protein kinase B; ORO: Oil Red O; BSA: bovine serum albumin; DMSO: dimethylsulfoxide; GTT: glucose tolerance test; AUC: area under the curve; HFD: high-fat diet; CT: control.

HRI Activators Increase HRI Phosphorylation in Human Huh-7 Hepatic Cells.

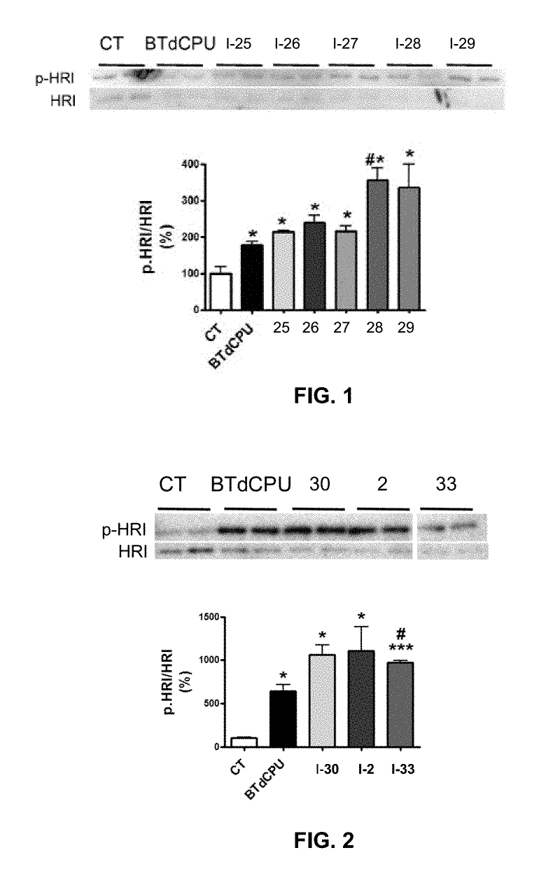

HRI is activated by autophosphorylation and this has been used for evaluating its activation. In fact, the hyperphosphorylation of HRI is accompanied by an increase of its eIF2α kinase activity (cf. Lu L. et al., “Translation initiation control by heme-regulated eukaryotic initiation factor 2a kinase in erythroid cells under cytoplasmic stresses”, Mol. Cell. Biol., 2001, vol. 21, pp. 7971-7980). Therefore, HRI protein constitutively exists as the native nonphosphorylated 76-kDa species and/or the autoactivated/autophosphorylated 92-kDa, although the relative abundance of each HRI species tends to vary depending on the cells examined (cf. Acharya P. et al., “Hepatic heme-regulated inhibitor (HRI) eukaryotic initiation factor 2a kinase: A protagonist of heme-mediated translational control of CYP2B enzymes and a modulator of basal endoplasmic reticulum stress tone”, Mol. Pharmacol, 2010, vol. 77, pp. 575-592). To evaluate whether the new synthesized compounds were able to activate HRI, inventors examined by Western-blot whether Huh-7 cells exposed to the new compounds showed an increase in the ratio phosphorylated/total HRI, indicating the activation of this enzyme by these compounds. Thus Huh-7 cells were incubated for 24 h with vehicle (DMSO, CT cells) or 10 μM of each of the assayed compound. The effects of these compounds were compared to the well-known HRI activator BTdCPU. Compounds I-25, I-26, I-27, I-28, and I-29 increased the ratio phosphorylated/total HRI (FIG. 1), especially compounds I-28 and I-29, indicating activation of this kinase. Compound I-30 (FIG. 2) also increased the levels of phosphorylated HRI similarly to those observed for BTdCPU, whereas the increase caused by I-33 was also statistically significant, mainly because of the reduction in total HRI caused by this compound.

HRI Activators Increase eIF2α Phosphorylation in Human Huh-7 Hepatic Cells.

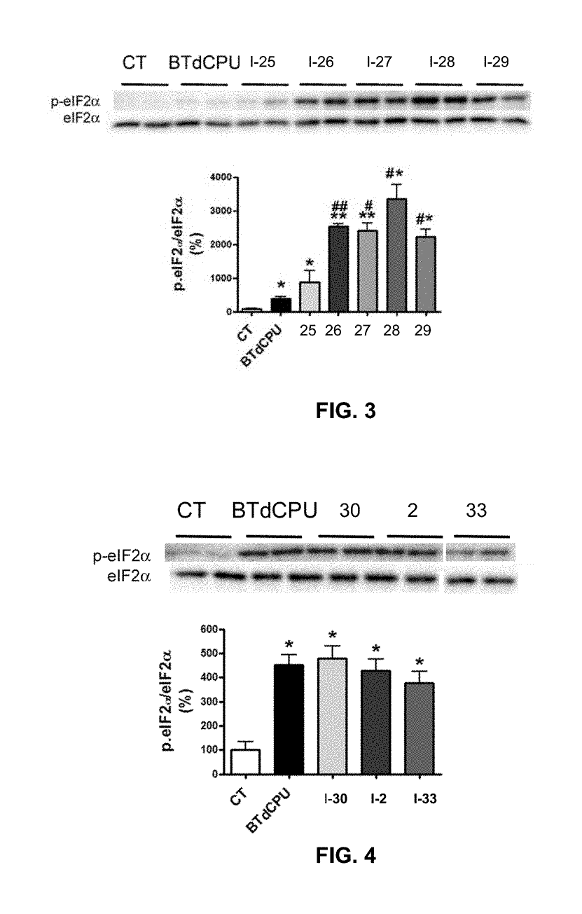

HRI is a kinase that phosphorylates eukaryotic Initiation Factor 2α (eIF-2a) at its Ser 51 residue to execute protein synthesis regulation and thereby HRI activators cause the phosphorylation of eIF2α as previously described (Chen T, et al., ibid). To confirm whether the new synthesized compounds were able to activate the HRI eIF2α kinase inventors examined the eIF2α phosphorylation caused by these compounds compared to the well-known HRI eIF2α kinase activator BTdCPU. Human Huh-7 hepatocytes were incubated for 24 h with vehicle (DMSO, CT cells) or 10 μM of each of the assayed compounds. eIF2α phosphorylation was determined by Western-blot. Compounds I-26, I-27, I-28, and I-29 caused a marked increase in eIF2α phosphorylation, higher than the observed with BTdCPU, whereas phosphorylation induced by compound I-25 was similar to that observed for BTdCPU (FIG. 3). Compounds I-30 and I-33 enhanced eIF2α phosphorylation to values similar to those achieved by BTdCPU exposure (FIG. 4).

Triglyceride Accumulation in Hepatocytes.

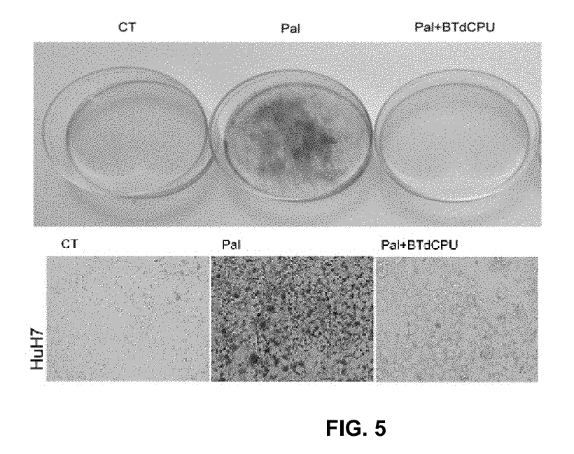

To examine the effects of HRI activators on triglyceride accumulation and insulin signaling in hepatocytes, the N,N′-diarylurea BTdCPU was used. HRI is a kinase that phosphorylates eIF2α at its Ser 51 residue to execute protein synthesis regulation and thereby HRI activators cause the phosphorylation of eIF2α as previously described (Chen T. et al., ibid). First, the effect of BTdCPU on human Huh-7 hepatocytes exposed to the saturated fatty acid palmitate was explored. Huh-7 cells were incubated for 24 h with BSA (Control, CT), 0.75 mmol/L palmitate (Pal) conjugated with BSA or 0.75 mmol/L palmitate plus 10 μmol/L BTdCPU (Pal+BTdCPU). Then, they were stained with Oil Red O that allows selective detection of neutral lipids (primarily triglyceride and cholesterol esters) within Huh-7 cells. Huh-7 cells exposed to palmitate showed a high accumulation of triglycerides, as demonstrated by Oil Red O (ORO) staining, but this accumulation was prevented in the presence of the BTdCPU compound (see FIG. 5). Thus, this assay demonstrates that HRI activation prevents palmitate-induced triglyceride accumulation in human Huh-7 hepatic cells.

Insulin Signalling in Hepatocytes.

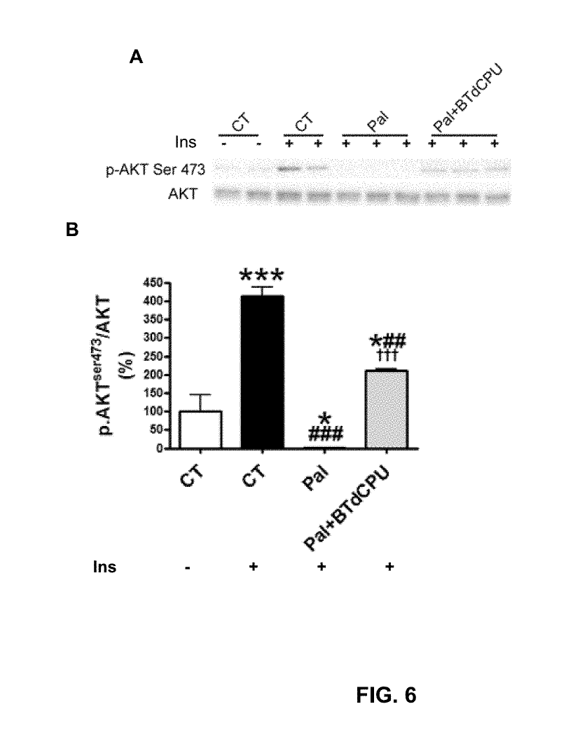

Inventors examined the insulin signalling pathway by measuring insulin-stimulated Akt phosphorylation. Huh-7 cells were incubated for 24 h with BSA (Control, CT), 0.75 mmol/L palmitate (Pal) conjugated with BSA, or 0.75 mmol/L palmitate plus 10 μmol/L BTdCPU (Pal+BTdCPU). Immunoblot analyses of total and phosphorylated Akt was carried out. FIG. 6 shows how, when cells were stimulated with insulin (positive control, CT+) for 10 min, this hormone increased the levels of phosphorylated Akt compared to control cells not exposed to insulin (negative control CT−). However, in cells exposed to the saturated fatty acid palmitate, insulin did not increase the phosphorylation of Akt, indicating that the insulin signalling pathway was attenuated. Interestingly, the BTdCPU compound partially restored the reduction in insulin-stimulated Akt phosphorylation caused by palmitate (see, FIG. 6), showing that this drug treatment prevents palmitate-induced insulin resistance. Thus, this assay demonstrates that HRI activation prevents palmitate-induced insulin resistance in human Huh-7 hepatic cells.

Glucose Tolerance.

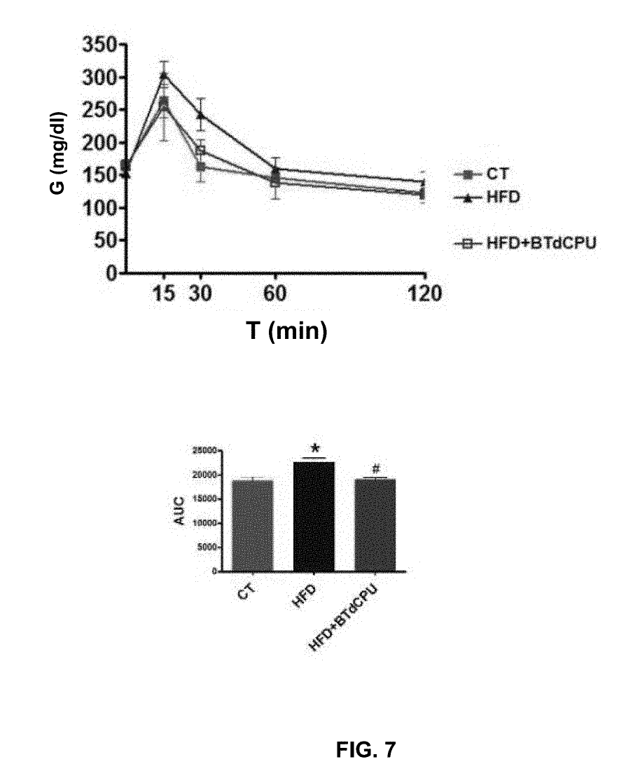

T2DM is characterized by glucose intolerance, which is contributed to by peripheral (muscle, fat, and liver) insulin resistance as well as islet 1-cell dysfunction (cf. Andrikopoulos S. et al., “Evaluating the glucose tolerance test in mice”, Am. J. Physiol. Endocrinol. Metab., 2008, vol. 295, pp. E1323-32). The glucose tolerance test (GTT) is used in clinical practice and research to identify individuals with impaired glucose tolerance by assessing the disposal of a glucose load. It is important to acknowledge that the GTT is the only means of identifying impaired glucose tolerance, which is considered a prediabetic state (cf. Andrikopoulos S. et al., ibid). The standard presentation of results from GTTs is a description of blood glucose levels over time after the glucose administration. Generally, a time course of absolute glucose levels is presented. When using diabetic or insulin-resistant models (such as the HFD-fed mouse), a time course of absolute glucose levels should still be presented along with a calculation of the AUC above baseline glucose. In this assay, the effect of the BTdCPU compound on glucose tolerance in mice fed a high-fat diet (HFD) was examined. For the assay, mice were fed a standard chow (CT), a HFD for three weeks (HFD), or a HFD for three weeks plus BTdCPU during the last week (HFD+BTdCPU). Mice fed a standard chow and half of the mice fed the HFD received one daily i.p. administration of DMSO (vehicle) for the last week. The rest of the mice fed the HFD received one daily i.p. administration of BTdCPU (70 mg kg⁻¹ day⁻¹) for the last week. As expected, the HFD significantly increased the AUC, indicating the presence of glucose intolerance (see FIG. 7). Of note, BTdCPU administration to mice fed the HFD prevented the increase in the AUC above baseline glucose and these mice showed an AUC similar to that observed in mice fed a standard diet (CT), demonstrating that BTdCPU prevents HFD-induced glucose intolerance. Thus, it is concluded that HRI activation prevents HFD-induced glucose intolerance.

Steatosis.

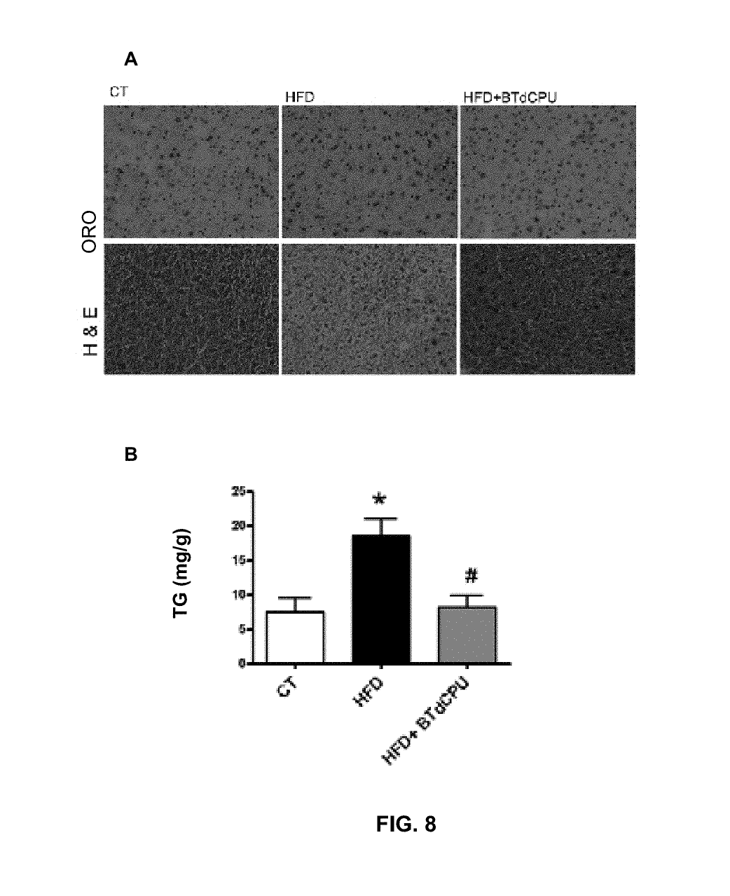

Inventors examined whether the treatment with an HRI activator (BTdCPU) prevented the development of steatosis induced by a HFD. Histological analysis of a liver biopsy remains the gold standard for assessing the degree of steatosis. ORO staining and eosin-hematoxylin (H&E) staining of liver sections were performed. Mice fed a standard chow and half of the mice fed the HFD received one daily i.p. administration of DMSO (vehicle) for the last week. The rest of the mice fed the HFD received one daily i.p. administration of BTdCPU (70 mg kg⁻¹ day⁻¹) for the last week. Compared to mice fed a standard diet, livers of mice fed a HFD showed the presence of fat droplets stained of red in the assessment of ORO sections (FIG. 8A, upper row). Similarly, analysis of H&E sections showed the presence of macrovesicular steatosis (FIG. 8A, lower row). When mice were fed with the HFD and treated with the HRI activator hepatic steatosis was reversed as demonstrated by ORO and H&E staining. Finally, hepatic lipids were extracted and hepatic triglyceride levels were assessed by using a commercially available kit (TR0100, Sigma). Livers of mice fed the HFD showed a significant increase in the levels of hepatic triglyceride and this increase was completely blunted when mice were treated with BTdCPU (FIG. 8B). Overall, these findings indicate that HRI activation prevents HFD-induced steatosis.

FGF21 Expression in Hepatocytes.

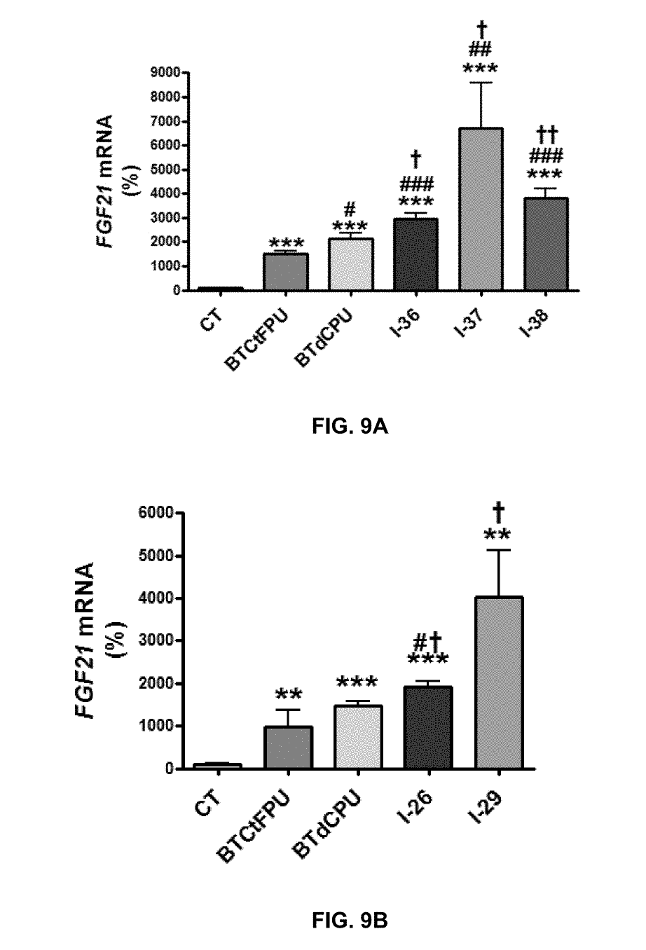

When inventors examined the effects of HRI activators on FGF21 expression in human Huh-7 hepatocytes they observed that compounds BTdCPU, I-36, I-37 and I-38 showed a significant higher increase than compound BTCtFPU. In addition, compounds I-36, I-37 and I-38 induced a significant higher increase in FGF21 expression than compound BTdCPU (cf. FIG. 9A). Moreover, treatment with compound I-26 led to a higher expression in FGF21 than BTCtFPU, whereas compounds I-26 and I-29 showed a higher increase in FGF21 expression than that observed for compound BTdCPU (cf. FIG. 9B).

### REFERENCES CITED IN THE APPLICATION

### Patent Documents

U.S. Pat. No. 3,073,861
U.S. Pat. No. 7,932,416
U.S. Pat. No. 8,937,088

### Non-Patent Documents

Acharya P. et al., “Hepatic heme-regulated inhibitor eukaryotic initiation factor 2a kinase: A protagonist of heme-mediated translational control of CYP2B enzymes and a modulator of basal endoplasmic reticulum stress tone”, Mol. Pharmacol, 2010, vol. 77, pp. 575-592.
Andrikopoulos S. et al., “Evaluating the glucose tolerance test in mice”, Am. J. Physiol. Endocrinol. Metab., 2008, vol. 295, pp. E1323-32.
Chen T. et al., “Chemical genetics identify eIF2α kinase heme-regulated inhibitor as an anticancer target”, Nature Chem. Biol., 2011, vol. 7, pp. 610-616, and Supp. Information.
Denoyelle S. et al., “In vitro inhibition of translation initiation by N,N′-diarylureas potential anti-cancer agents”, Bioorg. Med. Chem. Lett., 2012, vol. 22, pp. 402-409.
Karagiannidis L. E. et al., “Highly effective yet simple transmembrane anion transporter based upon ortho-phenylenediamine bis-ureas”, Chem. Commun., 2014. vol. 50, pp. 12050-12053, Electronic Supplementary Information.
Lu L. et al., “Translation initiation control by heme-regulated eukaryotic initiation factor 2a kinase in erythroid cells under cytoplasmic stresses”, Mol. Cell. Biol., 2001, vol. 21, pp. 7971-7980.
Marchesini G. et al., “Nonalcoholic fatty liver disease: a feature of the metabolic syndrome”, Diabetes, 2001, vol. 50, pp. 1844-1850.
Musso G. et al., “Metaanalysis: natural history of non-alcoholic fatty liver disease and diagnostic accuracy of non-invasive tests for liver disease severity”, Ann. Med., 2011 vol. 43, pp. 617-649.

1. A compound of formula (I) 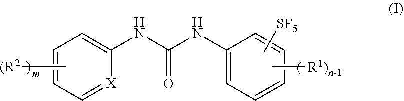
and pharmaceutically acceptable salts and solvates thereof, wherein:
X is CH or N;
R¹ is a radical independently selected from the group consisting of H, SF₅, halogen, CF₃, NO₂, CN, and OCF₃; n being an integer from 1 to 5; halogen being F, Cl, Br or I;
R² is a radical independently selected from the group consisting of SFs, halogen, CF₃, NO₂, CN, OCF₃, hydroxyl, (C₁-C₄)alkyl, (C₁-C₄)alkoxy, di-(C₁-C₄)alkylaminoethoxy, 2-(piperidin-1-yl)ethoxy, 2-(pyrrolidin-1-yl)ethoxy, 2-(azepan-1-yl)ethoxy, 2-morpholinoethoxy, and 2-(piperazin-1-yl)ethoxy; m being an integer from 0 to 5;
alternatively, when X is CH and m is an integer from 2 to 5, two R² radicals taken together with two adjacent carbons of the benzene ring to which they are joined form a 5- or 6-membered heterocyclic ring having from 1 to 3 heteroatoms independently selected from the group consisting of O, N, and S, the heterocyclic ring being fused with the benzene ring, and the benzene ring being optionally substituted with one or more radicals independently selected from the group consisting of SFs, halogen, CF₃, NO₂, CN, and OCF₃;
provided that the compound of formula (I) does not have any of the following formulas:

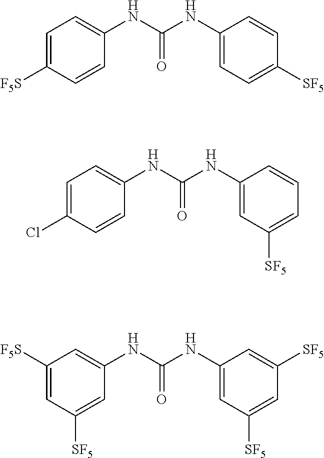

2. The compound according to claim 1, wherein X is CH.

3. The compound according to claim 1, wherein n is 1 or 2, and m is 1 or 2.

4. The compound according to claim 1, wherein: m is an integer from 2 to 5, and two R² radicals taken together with two adjacent carbons of the benzene ring to which they are joined form a 5- or 6-membered heterocyclic ring having from 1 to 3 heteroatoms independently selected from the group consisting of O, N, and S, the heterocyclic ring being fused with the benzene ring, and the benzene ring being optionally substituted with one or more radicals independently selected from the group consisting of SF₅, halogen, CF₃, NO₂, CN, and OCF₃.

5. The compound according to claim 4, wherein two R² radicals are forming a heterocyclic ring which has one of the following formulas:

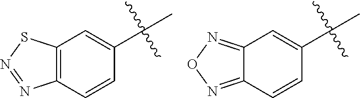 [SMILES: *c1ccc2nnsc2c1 ; *c1ccc2nonc2c1]

6. The compound according to claim 1, wherein R² is independently selected from the group consisting of SF₅, halogen, CF₃, NO₂, CN, OCF₃, hydroxyl, (C₁-C₄)alkyl, (C₁-C₄)alkoxy, di-(C₁-C₄)alkylaminoethoxy, 2-(piperidin-1-yl)ethoxy, 2-(pyrrolidin-1-yl)ethoxy, 2-(azepan-1-yl)ethoxy, 2-morpholinoethoxy, and 2-(piperazin-1-yl)ethoxy.

7. The compound according to claim 1, which has an SF₅ radical attached to the 3 position of the phenyl ring that has the R¹ radicals.

8. The compound according to claim 1, wherein halogen is F or Cl.

9. The compound according to claim 5, which has one of the following formulas:

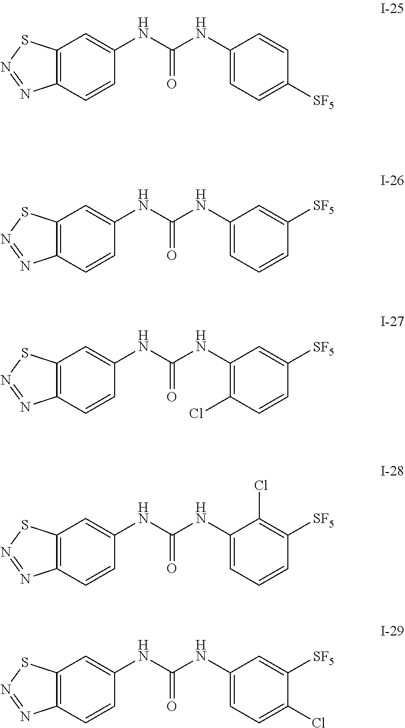

10. The compound according to claim 6, which has one of the following formulas:

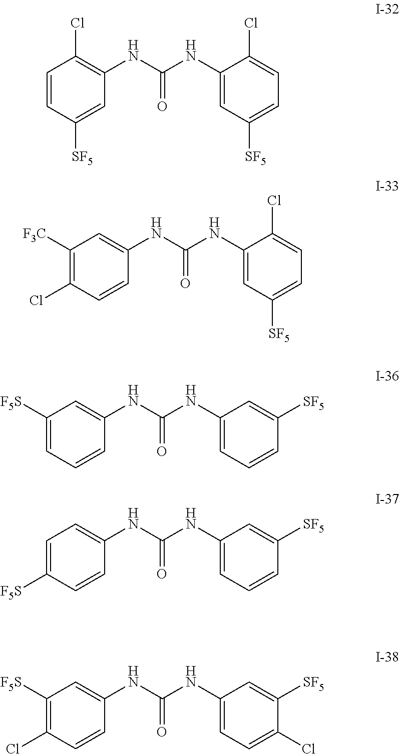

11. A compound according to claim 1, for use as active pharmaceutical ingredient.

12. A compound according to claim 1, for use in the prevention or treatment of a cardiometabolic disease selected from the group consisting of metabolic syndrome, obesity, insulin resistance, type 2 diabetes mellitus, non-alcoholic fatty liver disease, steatosis, non-alcoholic steatohepatitis, hypertension, dyslipidemia, atherosclerosis, and heart disease.

13. The compound for use according to claim 12, wherein the cardiometabolic disease is metabolic syndrome.

14. A pharmaceutical composition comprising a therapeutically effective amount of a compound according to claim 1, together with appropriate amounts of pharmaceutically acceptable excipients or carriers.

15. A pharmaceutical composition according to claim 14, for use in the prevention or treatment of a cardiometabolic disease selected from the group consisting of metabolic syndrome, obesity, insulin resistance, type 2 diabetes mellitus, non-alcoholic fatty liver disease, steatosis, non-alcoholic steatohepatitis, hypertension, dyslipidemia, atherosclerosis, and heart disease.
